(19) United States
(12) Patent Application Publication
(10) Pub. No.: US 2019/0151137 A1
(43) Pub. Date: May 23, 2019

(54) ANTI-OBSTRUCTIVE AIRWAY DENTAL ORTHOTIC HAVING MULTIPLE FIXED JAW DISPLACEMENT ADJUSTMENTS

(71) Applicant: James Metz, Columbus, OH (US)

(72) Inventor: James Metz, Columbus, OH (US)

(21) Appl. No.: 15/821,282

(22) Filed: Nov. 22, 2017

(51) Int. Cl. A61F 5/56; A61C 7/36; A61C 7/08

(52) U.S. Cl. CPC A61F 5/566; A61C 7/08; A61C 7/36

(57) ABSTRACT

An anti-obstructive airway dental orthotic (10) having multiple fixed jaw displacement adjustments is provided. A maxillary retainer (100) is connected to a mandibular retainer (200) by a strut having a length variable between a fixed minimum and a fixed maximum length. The maxillary retainer (100) has two pivot points (120, 125) such that the strut (300) may be moved from the second pivot point (125) to the first pivot point (120) to effect a progressive adjustment of mandibular advance. Additionally, the maxillary retainer (100) may be provided with a spacing area (140) that creates a minimum vertical separation between a cusp of the user's maxillary premolar and a cusp of the user's opposing mandibular premolar. Other fenestrations and cut-outs in the device (10) may also be provided.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD

The present disclosure relates generally to the field of dental orthotics, including a means for the advancement of the lower jaw, relative to the upper, and a means of achieving a minimum vertical separation of the maxillary and mandibular teeth, and in particular, to orthotics having multiple fixed-length jaw displacement adjustments.

### BACKGROUND OF THE INVENTION

A large number of persons have varying degrees of upper airway obstruction presenting with varied degrees of symptoms. As the upper airway begins with the nose and mouth, it is not surprising that the nose, tongue, and jaw all make contributions to such obstructive syndromes.

Various treatment modalities have been suggested. Various airway pressure devices, such as continuous positive airway pressure devices (CPAP) have been successfully used, although these tend to be both cumbersome and uncomfortable. More recently, dental orthotics have been employed. One general class of orthotic relies upon shifting the position of the lower jaw, generally moving the jaw forward, to displace the tongue anteriorly and thereby help clear the upper airway. Obviously, it is difficult to shift the jaw anteriorly without an external anchor point, so these orthotic have also presented problems of utility and comfort.

A classical means for advancement of the jaw is the Herbst appliance. The Herbst appliance is a fixed, tooth-borne, functional orthodontic appliance in which jaw position is influenced by a pin-and-tube spring-loaded appliance that is cemented or bonded to the teeth.

In particular, the Herbst appliance, or Herbst-type orthotics, often suffer from a number of practical problems. Generally, these devices are semi-permanently bonded to the teeth, and while they can be removed by a practitioner, they are not generally amenable to removal by the wearer. These appliances generally utilize spring-loaded struts, in order that a continuous pressure may be applied to advance the jaw.

Other appliances have adjustable displacements formed by either having an adjustable length strut, or by having an adjustable anchor point at one end of a strut, typically a slidable adjustment point, most often seen at the maxillary end of the strut. These devices depend on a practitioner making multiple adjustments, over time, to the degree of displacement of the jaw. The adjustable nature of these devices results in struts or adjustment points that are inherently complex and relatively weak.

### SUMMARY OF THE INVENTION

In its most general configuration, the presently disclosed dental orthotic advances the state of the art with a variety of new capabilities and overcomes many of the shortcomings of prior devices and methods in new and novel ways. In its most general sense, the presently disclosed dental orthotic also overcomes the shortcomings and limitations of the prior art in any of a number of generally effective configurations.

The assessment of a final desired jaw advancement may be made by a number of methods, which may include but are not limited to; subjective sense of airway improvement by the wearer, a measurement of a resting heart rate at an ambient atmosphere of the wearer, the achievement of an advancement to a predetermined amount of advancement by objective metrics, measuring and comparing resting arterial blood oxygen saturation level at an ambient atmosphere of the wearer to predetermined levels, measuring and comparing a resting heart rate at an ambient atmosphere of the wearer to predetermined levels, and perhaps most importantly, an assessment of obstructive sleep apnea events.

### SUMMARY OF THE INVENTION

Obstructive sleep apnea (OSA) is a prevalent and relatively underdiagnosed condition, which affects 12 to 22% of adults, and is characterized by recurrent episodes of partial and complete airway obstruction during sleep. As of 2012, 82% of men and 93% of women with OSA were estimated to be undiagnosed. The apnea-hypopnea index (AHI), as derived from the overnight polysomnogram, is the most commonly used measure of disease severity, with an AHI<5/hour being considered normal, 5-14.9/hour mild, 15-29.9/hour moderate, and ≥30 events/hour being defined as severe OSA. Patients with OSA are subjected to intermittent hypoxia, sympathetic activation, and sleep fragmentation, which if left untreated are independently associated with cardiometabolic disturbances, neurocognitive deficits, motor vehicle and work-related accidents, insomnia, anxiety and depression, sexual dysfunction, and ultimately, an increased risk for premature death.

Although polysomnography (PSG) is generally considered the “gold standard” diagnostic method, home sleep apnea testing (HSAT) via portable, unattended cardiorespiratory monitors may provide accurate diagnosis and is increasingly utilized. HSAT offers the benefit of cost-efficient diagnosis in the home sleep environment, as well as being recognized as not inferior to PSG.

Dental professionals can be an important part of the multidisciplinary sleep medicine team. The dental office can frequently provide an entry point into the healthcare system for those patients who are not regularly evaluated by a physician, and systematic OSA screening of dental patients can identify those who may warrant referral for suspected OSA. Clinical practice guidelines currently recommend using mandibular advancement devices (MAD) for adults with OSA who are intolerant of continuous positive airway pressure (CPAP) therapy, and dentists with sufficient training can readily provide such therapeutic intervention.

Historically, MADs have been employed to treat snoring and mild to moderate OSA. More recent evidence points to MADs being a viable option even for those patients with severe OSA, especially in cases of poor CPAP compliance. MADs have typically been shown to be less successful in the presence of concurrent obesity. A complete response (CR) to MAD therapy is defined as a post-treatment respiratory event index (REI) of <5 events/hour; existing literature demonstrates that typical CR rates hover around 35-40%.

However, as the studies behind this specification and the inventions resulting from it have shown, thinking exclusively in terms of MAD as a treatment for OSA may be missing part of the picture. In its simplest reduction, the oropharynx may be thought of as a box, whose principal space-occupying structure is the tongue. As has been informally said, the tongue is either in the mouth, or in the throat. Therefore, any intervention, including but not necessarily limited to MAD, that increases the volume of the box, will tend to decrease the chances for OSA. This will be shown to be an important consideration in the specification to follow, particularly but not exclusively, with reference to the concepts of minimizing orthotic size and minimum vertical jaw separation, neither of which is strictly related to mandibular advancement.

Numerous variations, modifications, alternatives, and alterations of the various preferred embodiments, processes, and methods may be used alone or in combination with one another as will become more readily apparent to those with skill in the art with reference to the following detailed description of the preferred embodiments and the accompanying figures and drawings.

### BRIEF DESCRIPTION OF THE DRAWINGS

Without limiting the scope of the dental orthotic strut as disclosed herein and referring now to the drawings and figures:

FIG. 1 is a front perspective view of an embodiment of an anti-obstructive airway dental orthotic device having multiple fixed-length jaw displacement adjustments;

FIG. 2 is a side perspective view of the dental orthotic device of FIG. 1;

FIG. 3 is a side perspective view of the dental orthotic device of FIGS. 1 and 2 shown with the strut removed;

FIG. 4 is a bottom (cephalic facing) view of an embodiment of a maxillary retainer of the dental orthotic device of FIGS. 1-3;

FIG. 5 is a top (caudal facing) view of a mandibular retainer of the anti-obstructive airway dental orthotic device of FIGS. 1-3.

FIG. 6 is a bottom (cephalic facing) view of another embodiment of a maxillary retainer of an anti-obstructive airway dental orthotic device; and

FIG. 7 is a top (caudal facing) view of another embodiment of a mandibular retainer of an anti-obstructive airway dental orthotic device.

These drawings are provided to assist in the understanding of the exemplary embodiments of the anti-obstructive airway dental orthotic having multiple fixed jaw advancement adjustments as described in more detail below and should not be construed as unduly limiting the dental orthotic. In particular, the relative spacing, positioning, sizing and dimensions of the various elements illustrated in the drawings are not drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

The disclosed anti-obstructive airway dental orthotic having multiple fixed jaw advancement adjustments enables a significant advance in the state of the art. The preferred embodiments of the dental orthotic accomplish this by new and novel arrangements of elements and methods that are configured in unique and novel ways and which demonstrate previously unavailable but preferred and desirable capabilities. The description set forth below in connection with the drawings is intended merely as a description of the presently preferred embodiments of the dental orthotic, and is not intended to represent the only form in which the dental orthotic may be constructed or utilized. The description sets forth the designs, functions, means, and methods of implementing the dental orthotic in connection with the illustrated embodiments. It is to be understood, however, that the same or equivalent functions and features may be accomplished by different embodiments that are also intended to be encompassed within the spirit and scope of the claimed dental orthotic.

As described throughout this specification the terms anterior and posterior shall describe relative positions to each other, and shall mean as follows: Anterior shall mean more distant from a coronal, or frontal plane, relative to the term posterior, which shall mean closer to a coronal, or frontal plane. The terms cephalic (towards the head) and caudal (towards the feet) shall have the usual medical meanings. The terms lingual (toward the tongue), buccal (towards the cheek), occlusal (towards the opposing tooth or teeth) shall have the usual meaning as applied in dentistry. Additionally, the term practitioner shall mean any person practicing the invention, which may be, by way of example and not limitation, any one of a wide variety of health care practitioners. The term “patient” or “user” shall mean any human subject employing the use of the device for any purpose whatsoever. One skilled in the art will realize the essentially variable nature of human physiology, and that therefore the use of the term “approximate” or “approximately” in the specification and claims must necessarily be subject to natural variation. However, it has been experienced that a range of plus or minus 20% will include the great majority of human variants, although this is not absolutely so. Accordingly, such a variation should be applied to all figures and measurements given.

### EXAMPLE 1

### Background

101 patients with polysomnographically (PSG) or HSAT (Home Sleep Apnea Testing)-diagnosed OSA who had either failed CPAP or who had refused CPAP therapy were evaluated over a 30-month span between May, 2013; and November, 2015 and completed the study. Written informed consent was obtained from each participant prior to initiating treatment. As indicated, OSA was clinically diagnosed via either PSG in an American Academy of Sleep Medicine (AASM) accredited sleep laboratory, or through HSAT (home sleep apnea testing) with a clinically recognized device. All studies were interpreted by a board-certified sleep medicine specialist.

Intake examinations consisted of medical, dental and sleep history, including a prescription of medical necessity for an oral appliance from the patient's sleep physician. Full-arch maxillary and mandibular impressions were acquired, along with a protrusive bite registration. A mandibular advancement device was fabricated for each patient, including both a maxillary and mandibular retainer, generally configured as dental trays. At insertion, each tray was verified for fit and comfort, and barrel-style strut (Herbst-style) left and right attachments, generally configured as variable length struts having a fixed minimum and maximum extensible length, were utilized to join the trays. Initial protrusion was set at 3.5 mm from where the teeth best fit together (habitual occlusion); starting minimum vertical dimension was set at 4 mm. The concept and importance of such minimum vertical dimension, or separation, is discussed below.

Patients were seen every 2-4 weeks to assess subjective symptom relief and side effects. After 6 to 8 weeks, high resolution pulse oximetry was employed for 3 nights with the MAD in place and results of this test served as a guide to the decision as to whether further titration and study nights were warranted. Lateral cephalometric radiography was employed to assess the impact of different amounts of vertical and horizontal dimension on the posterior airway space. Notably, involving the vertical dimension may allow for a reduced horizontal adjustment and minimize changes in occlusion. Average successful horizontal protrusion was considered as 7.5 mm and average optimal minimum vertical dimension was considered as 4 mm; the majority of the patients fell into these dimensions. Less minimum vertical dimension was used to promote lip competence with device usage among patients of small stature.

### Results

The final cohort that completed the MSAD treatment protocol included 101 adult OSA patients. Out of the 101, 77 (76.2%) had tried CPAP but could not tolerate it. Twenty (19.8%) had refused CPAP without attempting it, and 4 (4%) were comfortable with CPAP therapy, but requested a MAD as an alternate treatment, to use at their discretion, and used MAD exclusively during the study. Pre-MAD, the mean REI was 27.6/hour (SD 8.44) and the median was 17.3/hour (range: 5.0-90.3/hour). Forty-two (41.6%) subjects had mild OSA with REI between 5 and 14.9/hour; 21 (20.8%) had moderate OSA with REI between 15 and 29.9 events/hour; the remaining 38 subjects (37.6%) had severe OSA with REI≥30/hour.

Women comprised 41.6% (n=42) of the cohort. Mean age was 55.1 years old (SD 11.08) and median age was 57 years. Mean BMI was 30.6 kg/m² (SD 6.47) and median was 29.2 kg/m². Of the entire cohort, 12 (12.9%) were of normal weight with a BMI between 19.3 and 24.9; 42 (41.6%) were overweight with a BMI between 25 and 29.8 kg/m²; 37 (36.6%) were obese (BMI 30-39.9) and 9 (8.9%) were severely obese with BMI≥40 kg/m².

For the cohort, the overall reduction in REI after MAD was significant (p<0.001). Time-by-demographic variable interactions indicated that trends differed according to age (p=0.02) and gender (p=0.01), but not BMI (p=0.50). Older patients and men saw greater pre-post MAD reductions in REI, likely due to higher initial values.

Primary outcome was CR as defined by a post-treatment REI of <5 events/hour. Out of the 101 patients, 65 (64.3%) fulfilled CR criteria. Out of the remaining group, 23 (22.8%) had their REI drop>50% but remain above 5/hour, (i.e., Partial Responders or PR), while thirteen (12.9%) had their REI either not change or drop by less than 50% or, in rare cases, even go up (i.e., Non-Responders or NR). Almost half of the CR (32 or 49.2%) had mild OSA at baseline, but 23.1% (16) had moderate OSA, and 17 (29.2%) had severe OSA at baseline. The mean REI of the CR subjects was 21.8/hour (SD 17.05) and the median was 15.1. Eighty-six out of 101 (85.1%) attained an REI<10/hour, and 86/101 (85.1%) achieved a ≥50% reduction in REI.

The average nights of study of HRPO during appliance titration was 8; typically, three nights were performed at a time, but some nights were excluded due to loss of contact with the finger probe or loss of battery power. The average time from diagnostic PSG or HSAT to final efficacy clearing study was 364.71 days, with a range of 20 days at the minimum and 986 days as the maximum.

Mean age among CRs was 53.1 years (SD 10.99) and the median age was 57 years. Responders were significantly younger than NR (M=59.12, SD=10.48, p=0.01). CRs male/female ratio was approximately 1/1 (35 men and 32 women), though participating women had a higher proportion of CR and PR responders (78.05%) than men (58.33%, p=0.039). Responders' mean BMI was 29.89 kg/m² (SD 6.243) and the median was 28.8 kg/m². No significant differences existed in BMI between responders and NR (p=0.16). Responders did have a smaller mandibular advancement device protrusion (M=6.08 mm, SD=1.44) than NR (M=7.13 mm, SD=1.69, p=0.002), though no differences emerged in the minimum vertical component of the MAD devices (p=0.17).

Logistic regression examining potential predictors of favorable MAD response suggested that only age (OR=0.94, p=0.017) and protrusion of mandibular advancement device (OR=0.66, p=0.012) were significant predictors of MAD response. Neither gender (p=0.09), BMI (p=0.08), nor minimum vertical component of the device (p=0.63) were significant predictors, suggesting that these commonly cited predictors may not be valuable when adjusting for other variables. Additionally, although above pre-post analyses indicated gender differences in RDI trends, post-treatment REI values were quite similar, which may be reflected in the lack gender differences in odds of MAD response when adjusting for other variables.

### Discussion

Oral appliance therapy of OSA with a mandibular advancement device is an important treatment option among patients who are either intolerant of or reject CPAP. Studies have shown nearly half of those prescribed CPAP are non-adherent at one year of use, and 15 to 30% of those diagnosed with OSA reject CPAP without using it.

Substantial variability in the outcomes of MAD therapy has been reported, especially among severe OSA patients. Although the definition of CR or PR is not uniform across studies (common criteria used include for example: <5 events/hour; <10 events/hour; ‘favorable treatment response’ is >50% reduction of events/hour; ‘clinically significant decrease’ is <15/hour) and the reported sample sizes vary greatly (8-108 subjects), CR rates have traditionally hovered around between 11.1% and 40%. Not surprisingly, higher rates of CR occurred when a more lax definition of treatment success was used, i.e., 63.6% based on post-treatment outcome of <10 events/hour, and 62.5% based on post-treatment outcome of <15/hour.

The primary outcome implemented in the present study was a post-treatment reduction of the REI to <5 events/hour, which was achieved in 64.3% of the participants, with no significant differences detected across OSA severity categories. Similarly, as a comparison point to the aforementioned previous studies, 85.1% of subjects showed a post-treatment REI<10 events/hour. Here, the magnitude of horizontal protrusion but not the minimum vertical dimension, was found to be significantly associated with CR. These results suggest that the minimum vertical dimension selected falls into an anatomic norm, and furthering this dimension appears to yield no incremental benefits. Importantly, BMI did not emerge as a significant factor for MAD CR response; this is in stark contrast to previous studies suggesting that higher BMI contributes to reduced success with oral appliance therapy for OSA. The potential reasons for the discrepant findings are unclear, since our cohort consisted of consecutive patients and was not specifically selected for any of the a priori predictors of MAD unfavorable responses.

Among the major strengths of the present study, the inclusion of 101 subjects and the application of a stringent success criterion for CR, namely the reduction of REI to <5 events/hour are worthy of mention. Accordingly, the number of iterative overnight oximetry assessments and MAD adjustment sessions between the initial evaluation, and the final PSG or HSAT could have been much higher. This was not the case however, whereby the mean number of intermediary sessions and total duration of MAD titration in CR, PR and NR were remarkably similar, indicating that the comparatively favorable CR rates in this cohort were not the result of more prolonged and labor intensive efforts, but rather reflect individual factors across CR and non-CR, namely age and degree of horizontal protrusion achieved. Thus, implementation of a simple and low cost titration method, such as the home use of a high resolution pulse oximeter which allows for multiple nights of study with relative ease and minimal expense to the patient and medical provider, may facilitate more objective adjustments to be made in the process of MAD titration, ultimately resulting in more favorable CR rates.

### Conclusion

Significant improvements in OSA severity were apparent in patients at all disease severity levels. In sixty-seven subjects (66.3%) a respiratory event index (REI)<5 events/hour occurred, with 86 patients (85.1%) achieving a post-treatment REI<10 events/hour. In a stepwise logistic regression model, predictors of residual OSA included age (OR=0.94, p=0.017) and protrusion of mandibular advancement device (OR=0.66, p=0.012).

OSA at any level of severity can be effectively treated with a mandibular advancement device, even in the presence of obesity, with age and horizontal protrusion emerging as the only two predictive factors associated with CR. Meticulous attention to the titration process with high resolution pulse oximetry testing appears to be an important contributor to guide the titration process of MAD, and optimize CR rates. Mandibular advancement devices are typically comfortable for the patient when properly fabricated and adjusted, and therefore appear to be associated with high adherence. Mandibular advancement is an important option when positive airway pressure therapy is either unsuccessful or rejected

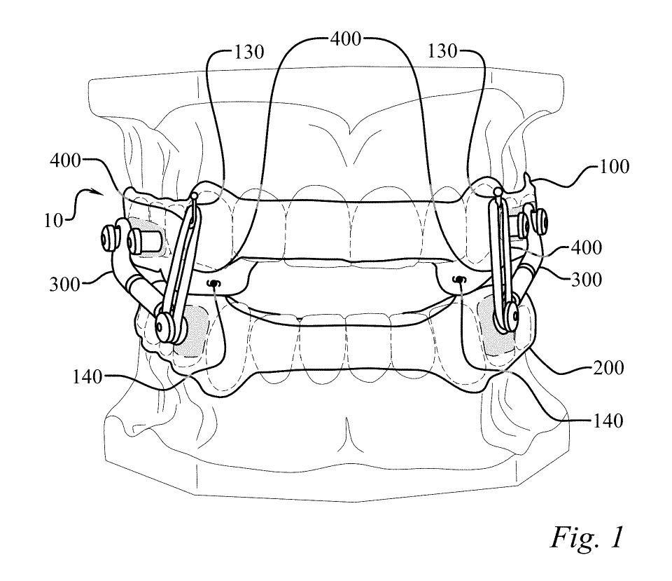

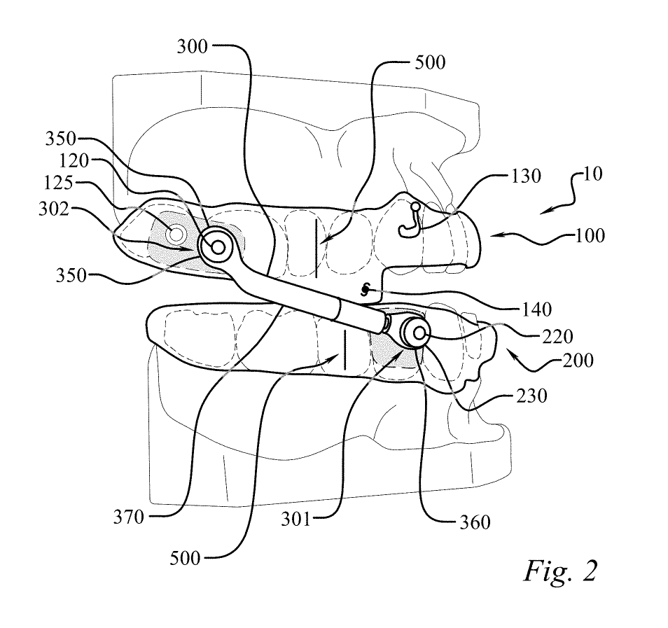

What is claimed then, and seen well in FIGS. 1-7, is an anti-obstructive airway dental orthotic (10), with a rigid strut (300) having a strut body (370), seen well in FIGS. 1-2, of variable length extendable between a predetermined minimum length and a predetermined maximum length. This has marked advantages over the adjustable struts that are typical in prior art devices. Adjustable length struts are more complex, have more parts, and these parts are necessarily more delicate and therefore weaker. The strut (370) of the instant invention may have an anterior end (301) having a rotable strut to mandible pivot (360), and a posterior end (302) having a rotable strut to maxilla pivot (350), as seen well in FIG. 2.

The device (10) also includes a maxillary retainer (100) for cooperating with and reversibly attaching to a plurality of maxillary teeth of a user, with the maxillary retainer (100) including a first maxilla to strut pivot point (120) and a second maxilla to strut pivot point (125), as seen well in FIG. 2.

Lastly, and also as seen well in FIGS. 1-22, the device (10) includes a mandibular retainer (200) for cooperating with and reversibly attaching to a plurality of mandibular teeth of the user, and the mandibular retainer (200) includes a mandible to strut pivot point (220).

So, and again as seen well in FIG. 2, fully assembled, the strut to maxilla pivot (350) rotably cooperates with at least one of the first maxilla to strut pivot point (120) and the second maxilla to strut pivot point (125) to rotably and reversibly connect the maxillary retainer (100) and the strut (300). The strut to mandible pivot (360) rotably cooperates with the mandible to strut pivot point (220) to rotably and reversibly connect the mandibular retainer (200) and the strut (300). Thus, the maxillary retainer (100) and the mandibular retainer (200) are reversibly and rotably joined by a strut (300) having a fixed minimum and maximum length to form the finished device (10).

The strut, having a fixed minimum and maximum length is a conventional barrel-type strut, similar to that seen in the well-known Herbst appliance. However, there is no spring or other tension internally produced by the strut. The barrel, or piston, is simply allowed to slide (expand and contract in length) within minimum and maximum parameters by tension or compression applied at the end of the strut. Mandibular advancement is accomplished by the fact that the strut (300) is simply slightly longer than the space between the pivot points (120, 125 and 220) in a position of habitual occlusion. In fact, no particular construction of the strut is required, other than it rotably and reversibly join the maxillary retainer (100) and the mandibular retainer (200), and that it move freely with the above-mentioned length parameters.

At least one of the pivot points, the first maxilla to strut pivot point (120), the second maxilla to strut pivot point (125) and the mandible to strut pivot point (220) may be attached to a reinforcement attached to the orthotic (10), as seen well in FIGS. 2-7. In some embodiments, this reinforcement includes at a maxillary reinforcement (110) and a mandibular reinforcement (210), and even more typically, there are reinforcements underlying each pivot point (120, 125, 220).

There may be a wide variety of reinforcements employed. In some embodiments, the reinforcement may be a metal reinforcement and/or an aramid fiber reinforcement. In other embodiments, the reinforcement may further be composed of steel and molybdenum. This may be in the form of a low carbon steel alloy, which is noteworthy as such are unusual alloys for dental use.

In one particular set of embodiments, the reinforcements may be an L-shaped reinforcement embedded within at least one of the retainers and having both an occlusal limb and a buccal limb. This has been shown to have major advantages in the fabrication of the device (10). Computer mapping of the patient's jaw can indicate the precise place where an inset should be cast, milled, or otherwise formed into the outside top or bottom and outside (relative to the in-use orientation of the device) of the retainer(s). Then, the L-shaped retainer may simply be placed in the inset area, where it may be embedded in plastic, adhered, or in some other way firmly attached to the device (10). It is believed that the advent of 3-D printing will allow such reinforcement to be directly emplaced and printed within the actual mass of the retainer.

Thus, in some embodiments, the reinforcement can include both a maxillary reinforcement (110) and a mandibular reinforcement (210), seen well in FIGS. 2-7, and both reinforcements may configured as substantially L-shaped reinforcements each having an occlusal limb and a buccal limb.

The first maxilla to strut pivot point (120), the second maxilla to strut pivot point (125), and the mandible to strut pivot point (220), all seen well in FIG. 2, each have a central axis of rotation about the respective pivot point (120, 125, 220), and all central axes of rotation lie in parallel planes. This is important to allow free rotation of the strut (300) without binding upon the pivot points (120, 125, 220).

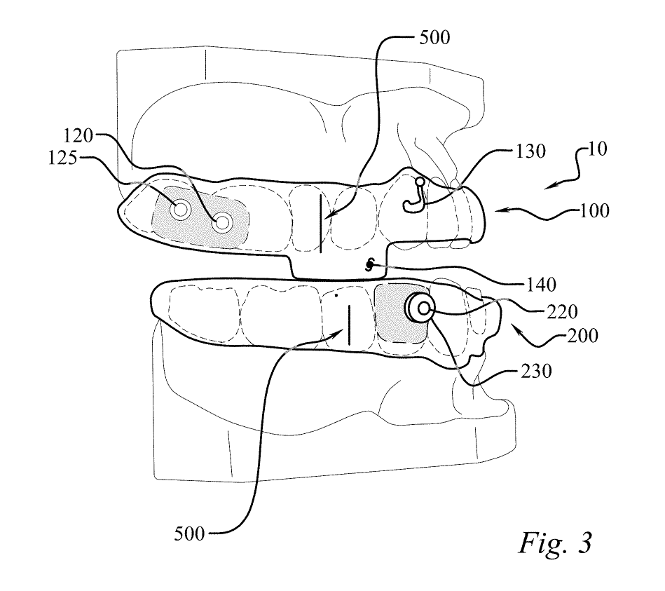

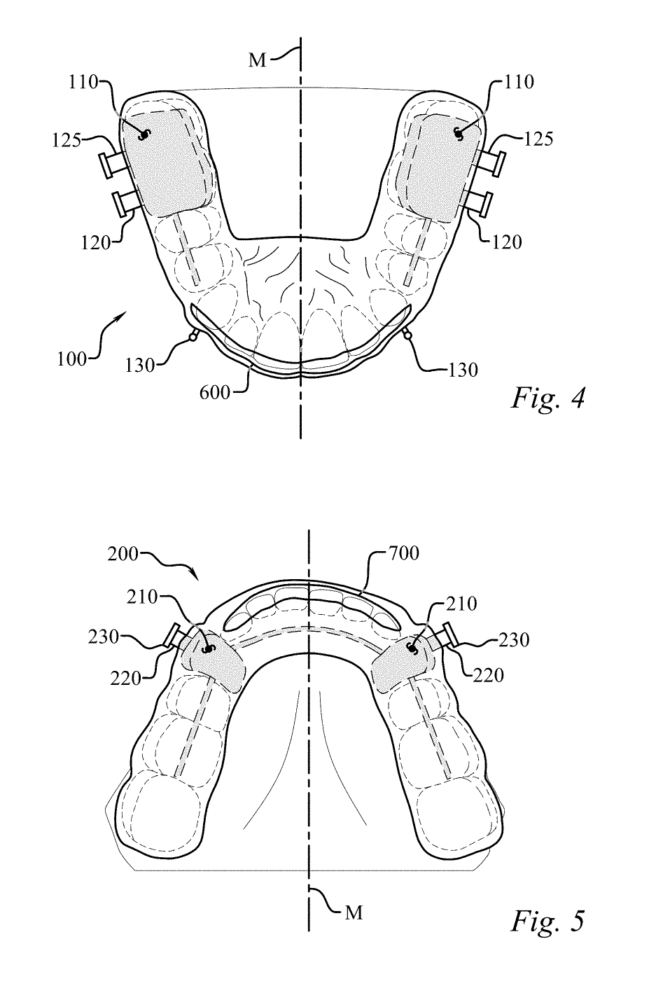

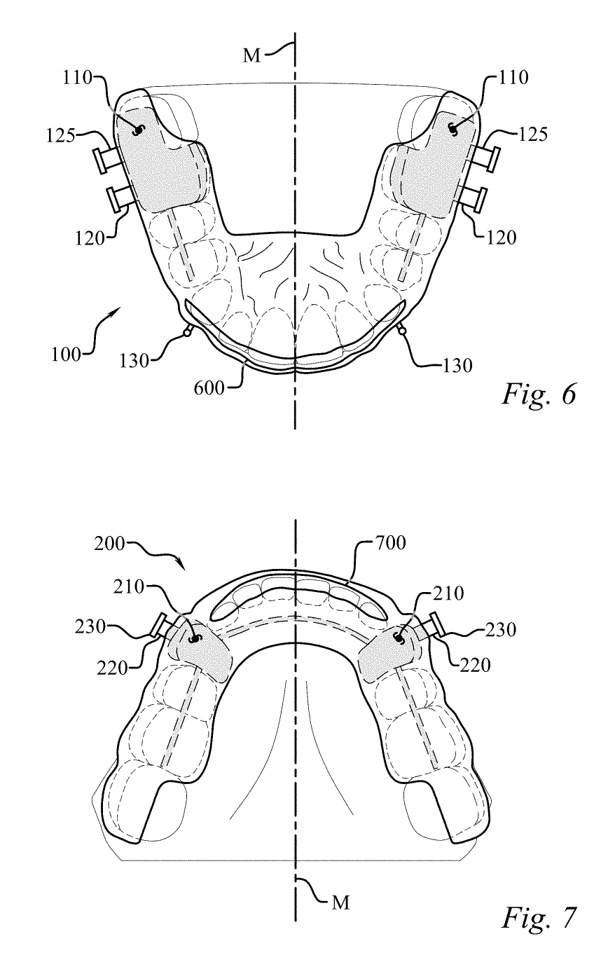

Unlike prior art devices where the jaw is advanced either by substituting a longer strut or by moving (generally releasing and then sliding an adjustable anchor on the maxillary retainer) a pivot point forward, many embodiments of the present device function to accomplish advancement beyond an initial position by moving the strut (300) from a more posterior to a more anterior pivot point (120, 125), i.e., by moving the strut (300) from the second maxillary pivot point (125) to the first maxillary pivot point (120). The central axis of the rotation of the first maxilla to strut pivot point (120) and the second maxilla to strut pivot point (125), seen well in FIGS. 2-3, each lie in a plane such that the central axes of both the first maxilla to strut pivot point (120) and the second maxilla to strut pivot point (125) are each equidistant from a plane of an occlusal surface of the maxillary retainer (100). The first maxilla to strut pivot point (120) and the second maxilla to strut pivot point (125) may have an anterior-posterior separation, seen well in FIGS. 4 and 6, measured along an imaginary line drawn parallel to an occlusal surface of the maxillary retainer (100) of about 4 millimeters, to allow for a corresponding advancement of the jaw.

In yet other embodiments, the maxillary retainer (100) and/or the mandibular retainer (200) may have an interior buccal surface, and interior lingual surface, and an interior occlusal surface, as would be known to one skilled in the art of traditional dental trays. In many prior art devices, the interior occlusal surface is molded to the individual tooth contour of a user. However, in certain embodiments of the present device, the interior occlusal surface i.e., the occlusal surface of the interior of the retainer, may be substantially flat and in use, simply contact a plurality of tooth occlusal surfaces. This has been found to be efficacious, and save a step, and the resultant costs, involved in a more complex molding process.

The study referenced above demonstrated the importance of minimum vertical dimension, or separation, in addition to mandibular advancement in relieving OSA. In many embodiments, the maxillary retainer (100) may have a spacing section (140), seen well in FIGS. 1-3, having an area of increased thickness such that in use, a minimum vertical separation is produced between a cusp of the user's maxillary premolar and a cusp of the user's opposing mandibular premolar of approximately between 2 and 4 millimeters. FIG. 3 shows a lateral view of the orthotic (10) with the strut (300) not shown, to better illustrate this spacing section (140).

This thickened spacing section (140) is in marked difference to the general goal of making the thickness of the retainer (100, 200) portions as thin as possible. In particular, the buccal portion of the retainers (100, 200) is ideally held to a thickness optimally not greater than 1.0 mm, in order to preserve as much intraoral space for the tongue as possible. It may be possible, using strong lightweight materials, to reduce this thickness to 0.5 mm. In a particular set of embodiments, also well seen in FIG. 3, the maxillary retainer (100) has a spacing section (140) comprising an area of increased thickness such that in use, a minimum vertical separation is produced between a cusp of the user's maxillary premolar and a cusp of the user's opposing mandibular premolar of approximately 4 millimeters. It has been found in clinical testing that a 4 mm minimum vertical separation is appropriate for approximately 80% of all patients, with only approximately 20% requiring a spacing outside of this parameter. Smaller advancements are most typically seen in a small number of patients of small stature who are unable to achieve an adequate “lip seal” with a greater minimum vertical separation. The suitability of a 4 mm minimum vertical separation in a large percentage of patients is both an unexpected discovery and a great improvement in orthotic fabrication. Rather than beginning with a “blank slate” of options in choosing minimum vertical separation, a practitioner can know that a very large percentage of patients' needs will cluster around a relatively specific spacing.

In a further subset of embodiments, the maxillary retainer (100) has a spacing section (140) comprising an area of increased thickness that extends laterally from approximately the anterior-posterior midline of the teeth to a buccal margin of the maxillary retainer (100). Similarly, the maxillary retainer (100) may have a spacing section (140) comprising an area of increased thickness that extends anterior-posterior across occlusal surfaces of only at least a first maxillary premolar and a second maxillary premolar of the user. Not having the spacing section (140) cover the entire occlusal surface of more teeth than necessary promotes both the goals of reserving maximum free space within the oral cavity for the tongue, and easing the fitting process for both the user and practitioner.

When the device (10) is fully assembled and in use, and when the strut to maxilla pivot (350) is attached to either the first maxilla to strut pivot point (120) or the second maxilla to strut pivot point (125), and the strut to mandible pivot (360) is attached to the mandible to strut pivot point (220), an anterior mandible advancement beyond a point of habitual occlusion of approximately 3 to 8 millimeters is produced, as seen well in FIG. 2. In a particular set of embodiments, when the devices (10) is fully assembled and in use, and when the strut to maxilla pivot (350) is attached to the second maxilla to strut pivot point (125) and the strut to mandible pivot (360) is attached to the mandible to strut pivot point (220), an anterior mandible advancement beyond a point of habitual occlusion of approximately 3.5 millimeters is produced during use.

However, in the most common configuration clinically employed, by way of example and not limitation only, when the device (10) is fully assembled and in use, and when the strut to maxilla pivot (350) is attached to the first maxilla to strut pivot point (120) and the strut to mandible pivot (360) is attached to the mandible to strut pivot point (220), an anterior mandible advancement beyond a point of habitual occlusion of approximately 7.5 millimeters is produced. Again, as described above, this is suitable for a large percentage of patients, thereby obviating the misperceived need for continuous and multiple adjustments of advancement with an adjustable strut. Again, it also removes the “blank slate” of possible advancements presented to the practitioner in the majority of cases, and eases the fitting process for both the user and practitioner.

In some embodiments, the orthotic (10) may have at least one fenestration, selected from the group of fenestrations consisting of a maxillary fenestration (600) seen well in FIGS. 4 and 6, and a mandibular fenestration (700), seen well in FIGS. 5 and 7, allowing the exposure of at least one incisal surface of at least one tooth through the orthotic (10). Again, this subserves the function of allowing maximal expansive area for the tongue.

In some embodiments, a portion a portion of the lingual and occlusal surface of at least one molar, selected from the group of molars consisting of the left first molar, the left second molar, the right first molar and the right second molar is not covered by the orthotic (10), as partially seen in FIGS. 6 and 7. In clinical practice, and in some subsets of patients only, this is generally configured as having the lingual surface and approximately ⅓ to ½ of the occlusal surface of all four second molars so exposed, and has led to a marked clinical improvement in these subsets.

Accordingly, as seen well in FIGS. 1-7, and omitting further redundant references to the Figures, an anti-obstructive airway dental orthotic (10) can include a rigid strut (300) having a strut body (370) of variable length extendable between a predetermined minimum length and a predetermined maximum length. The strut (300) may have an anterior end (301) having a rotable strut to mandible pivot (360), and a posterior end (302) having a rotable strut to maxilla pivot (350).

There may be a maxillary retainer (100) for cooperating with and reversibly attaching to a plurality of maxillary teeth of a user, having a spacing section (140) that has an area of increased thickness such that in use, a minimum vertical separation of approximately between 2 and 4 millimeters is produced between a cusp of the user's maxillary premolar and a cusp of the user's opposing mandibular premolar. The maxillary retainer (100) can also include a first maxilla to strut pivot point (120) and a second maxilla to strut pivot point (125), each having a central axis of rotation about the respective pivot point (120, 125). The pivot points (120, 125) may have an anterior-posterior separation of about 4 millimeters measured center-to-center along an imaginary line drawn parallel to an occlusal surface of the maxillary retainer (100), and be lying in a plane such that the central axes of both pivot points (120, 125) are each equidistant from the plane of the occlusal surface of the maxillary retainer (100).

The device (10) may include a mandibular retainer (200) for cooperating with and reversibly attaching to a plurality of mandibular teeth of the user, wherein the mandibular retainer (200) includes a mandible to strut pivot point (220) having a central axis of rotation about the respective pivot point (220). In some embodiments, the central axis of rotation of the first maxilla to strut pivot point (120), the second maxilla to strut pivot point (125), and the mandible to strut pivot point (220) all lie in parallel planes. The strut to maxilla pivot (350) may rotably cooperate with at least one of the first maxilla to strut pivot point (120) and the second maxilla to strut pivot point (125) to rotably and reversibly connect the maxillary retainer (100) and the strut (300). The strut to mandible pivot (360) may also rotably cooperate with the mandible to strut pivot point (220) to rotably and reversibly connect the mandibular retainer (200) and the strut (300), such that in use, an anterior mandible advancement beyond a point of habitual occlusion of approximately between 3 and 8 millimeters is produced.

In an additional series of embodiments the maxillary retainer (100) spacing section (140) may have an area of increased thickness such that in use, a minimum vertical separation is produced between a cusp of the user's maxillary premolar and a cusp of the user's opposing mandibular premolar of approximately 4 millimeters. As noted previously, this is an appropriate separation for a very large majority of users. In yet another series of embodiments, when the device (10) is fully assembled and in use, an anterior mandible advancement beyond the point of habitual occlusion of approximately 7.5 millimeters is produced. Once again, this is an appropriate advancement for a very large majority of users.

Such a system may be invoked, by means of example and not limitation only, by steps that may include, first, performing a baseline evaluation of a patient's medical, dental and sleep history, then forming an anti-obstructive airway dental orthotic (10) for that patient's use. Such a device (10) may have a rigid strut (300) having a strut body (370) of variable length extendable between a predetermined minimum length and a predetermined maximum length, an anterior end (301) having a rotable strut to mandible pivot (360), and a posterior end (302) having a rotable strut to maxilla pivot (350). There may be a maxillary retainer (100) included for cooperating with and reversibly attaching to a plurality of maxillary teeth of a user, having a spacing section (140) with an area of increased thickness such that in use, a minimum vertical separation of approximately 4 millimeters is produced between a cusp of the user's maxillary premolar and a cusp of the user's opposing mandibular premolar. The maxillary retainer (100) may include a first maxilla to strut pivot point (120) and a second maxilla to strut pivot point (125), each having a central axis of rotation about the respective pivot point (120, 125), and having an anterior-posterior separation of about 4 millimeters measured center-to-center along an imaginary line drawn parallel to an occlusal surface of the maxillary retainer (100). The pivot points (120, 125) may lie in a plane such that the central axes of both pivot points (120, 125) are each equidistant from the plane of the occlusal surface of the maxillary retainer (100).

There may be a mandibular retainer (200) for cooperating with and reversibly attaching to a plurality of mandibular teeth of the user, wherein the mandibular retainer (200) includes a mandible to strut pivot point (220) having a central axis of rotation about the respective pivot point (220). The central axis of rotation of the at least a first maxilla to strut pivot point (120), the at least a second maxilla to strut pivot point (125), and the mandible to strut pivot point (220) all lie in parallel planes, all to facilitate smooth operation of the device (10).

The strut to maxilla pivot (350) may initially rotably cooperate with the second maxilla to strut pivot point (125) to rotably and reversibly connect the maxillary retainer (100) and the strut (300), and the strut to mandible pivot (360) may rotably cooperate with the mandible to strut pivot point (220). Thus, the maxillary retainers (100), the mandibular retainer (200) and the strut (300), are all connected such that when the device is fully assembled and in use, with the maxillary retainer (100) cooperating with and reversibly attached to the plurality of maxillary teeth of the user and the mandibular retainer (200) cooperating with and reversibly attached to the plurality of mandibular teeth of the user, an anterior mandible advancement of approximately 4 millimeters from a point of habitual occlusion is produced.

The orthotic (10) may then be reversibly applied to the maxillary and mandibular teeth of the patient and the practitioner would verify the fit and comfort of the orthotic (10) to the patient. The next step would be waiting a predetermined period of time and making periodic assessments of the constellation of the patient's subjective symptomology, as well as waiting a predetermined period of time, and making an assessment of the constellation of the patient's objective symptomology.

As part of a titration process attuned to the subjective and objective indices, the practitioner could evaluate and adjust the thickness of the spacing area (140), and potentially move the strut (300) from the second maxilla to strut pivot point (125) to the first maxilla to strut pivot point (120), such that in use, with the maxillary retainer (100) cooperating with and reversibly attached to the plurality of maxillary teeth of the user and the mandibular retainer (200) cooperating with and reversibly attached to the plurality of mandibular teeth of the user, an anterior mandible advancement of approximately 7.5 millimeters from the point of habitual occlusion is produced

Then, the process could involve waiting a predetermined period of time and again making periodic assessments of the constellation of the patient's subjective and objective symptomology, and then the steps above may be repeated as required for a satisfactory relief of symptomology.

One skilled in the art will know that the assessment of a final desired jaw advancement may be made with a number of methods, which may include but are not limited to; subjective sense of airway improvement by the wearer, a measurement of a resting heart rate at an ambient atmosphere of the wearer, the achievement of an advancement to a predetermined amount of advancement by objective metrics, measuring and comparing resting arterial blood oxygen saturation level at an ambient atmosphere of the wearer to predetermined levels, and/or measuring and comparing a resting heart rate at an ambient atmosphere of the wearer to predetermined levels, and perhaps most importantly, evaluation of sleep apnea events.

Numerous alterations, modifications, and variations of the preferred embodiments disclosed herein will be apparent to those skilled in the art and they are all anticipated and contemplated to be within the spirit and scope of the disclosed anti-obstructive airway dental orthotic having multiple fixed jaw displacement adjustments (10). For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the dental orthotic (10) are described herein, it is to be understood that the practice of such additional modifications and variations and the equivalents thereof, are within the spirit and scope of the dental orthotic (10) as disclosed herein. The corresponding structures, materials, acts, and equivalents of all means or step plus function elements in the claims below are intended to include any structure, material, or acts for performing the functions in combination with other claimed elements as specifically claimed.

I claim:

1. An anti-obstructive airway dental orthotic (10), comprising:
a rigid strut (300) having a strut body (370) of variable length extendable between a predetermined minimum length and a predetermined maximum length, an anterior end (301) having a rotable strut to mandible pivot (360), and a posterior end (302) having a rotable strut to maxilla pivot (350),
a maxillary retainer (100) for cooperating with and reversibly attaching to a plurality of maxillary teeth of a user, wherein the maxillary retainer (100) includes a first maxilla to strut pivot point (120) and a second maxilla to strut pivot point (125),
a mandibular retainer (200) for cooperating with and reversibly attaching to a plurality of mandibular teeth of the user, wherein the mandibular retainer (200) includes a mandible to strut pivot point (220), and wherein
the strut to maxilla pivot (350) rotably cooperates with at least one of the first maxilla to strut pivot point (120) and the second maxilla to strut pivot point (125) to rotably and reversibly connect the maxillary retainer (100) and the strut (300), and
the strut to mandible pivot (360) rotably cooperates with the mandible to strut pivot point (220) to rotably and reversibly connect the mandibular retainer (200) and the strut (300).

2. The device according to claim 1, wherein at least one of the pivot points selected from the group of pivot points consisting of the first maxilla to strut pivot point (120), the second maxilla to strut pivot point (125) and the mandible to strut pivot point (220) is attached to a reinforcement attached to the orthotic (10).

3. The device according to claim 2, wherein the reinforcement comprises at least one of a maxillary reinforcement (110) and a mandibular reinforcement (210).

4. The device according to claim 2, wherein the reinforcement is selected from the group of reinforcements consisting of a metal reinforcement and an aramid fiber reinforcement.

5. The device according to claim 2, wherein the reinforcement further comprises steel and molybdenum.

6. The device according to claim 3, wherein at least one of the reinforcements is an L-shaped reinforcement embedded within at least one of the retainers consisting of the maxillary retainer (100) and the mandibular retainer (200), and is a substantially L-shaped reinforcement having an occlusal limb and a buccal limb.

7. The device according to claim 3, wherein the reinforcement comprises both a maxillary reinforcement (110) and a mandibular reinforcement (210), and both reinforcements are configured as substantially L-shaped reinforcements each having an occlusal limb and a buccal limb.

8. The device according to claim 1, wherein the first maxilla to strut pivot point (120), the second maxilla to strut pivot point (125), and the mandible to strut pivot point (220) each have a central axis of rotation about the respective pivot point (120, 125, 220), and all central axes of rotation lie in parallel planes.

9. The device according to claim 8, wherein the central axis of the rotation of the first maxilla to strut pivot point (120) and the second maxilla to strut pivot point (125) lie in a plane such that the central axes of both the first maxilla to strut pivot point (120) and the second maxilla to strut pivot point (125) are each equidistant from a plane of an occlusal surface of the maxillary retainer (100).

10. The device according to claim 1, wherein the first maxilla to strut pivot point (120) and the second maxilla to strut pivot point (125) have an anterior-posterior separation, measured along an imaginary line drawn parallel to an occlusal surface of the maxillary retainer (100) of about 4 millimeters.

11. The device according to claim 1, wherein at least one of the maxillary retainer (100) and the mandibular retainer (200) has an interior buccal surface, an interior lingual surface, and an interior occlusal surface, wherein the interior occlusal surface is substantially flat, and in use contacts a plurality of tooth occlusal surfaces.

12. The device according to claim 1, wherein the maxillary retainer (100) has a spacing section (140) comprising an area of increased thickness such that in use, a minimum vertical separation is produced between a cusp of the user's maxillary premolar and a cusp of the user's opposing mandibular premolar of approximately between 2 and 4 millimeters.

13. The device according to claim 1, wherein the maxillary retainer (100) has a spacing section (140) comprising an area of increased thickness such that in use, a minimum vertical separation is produced between a cusp of the user's maxillary premolar and a cusp of the user's opposing mandibular premolar of approximately 4 millimeters.

14. The device according to claim 1, wherein the maxillary retainer (100) has a spacing section (140) comprising an area of increased thickness that extends laterally from approximately an anterior-posterior midline of the teeth to a buccal margin of the maxillary retainer (100).

15. The device according to claim 1, wherein the maxillary retainer (100) has a spacing section (140) comprising an area of increased thickness that extends anterior-posterior across the occlusal surfaces of only at least a first maxillary premolar and a second maxillary premolar of the user.

16. The device according to claim 1, wherein when the strut to maxilla pivot (350) is attached to one of the first maxilla to strut pivot point (120) and the second maxilla to strut pivot point (125), and the strut to mandible pivot (360) is attached to the mandible to strut pivot point (220), an anterior mandible advancement beyond a point of habitual occlusion of approximately 3 to 8 millimeters is produced during use.

17. The device according to claim 1, wherein when the strut to maxilla pivot (350) is attached to the second maxilla to strut pivot point (125) and the strut to mandible pivot (360) is attached to the mandible to strut pivot point (220), an anterior mandible advancement beyond a point of habitual occlusion of approximately 3.5 millimeters is produced during use.

18. The device according to claim 1, wherein when the strut to maxilla pivot (350) is attached to the first maxilla to strut pivot point (120) and the strut to mandible pivot (360) is attached to the mandible to strut pivot point (220), an anterior mandible advancement beyond a point of habitual occlusion of approximately 7.5 millimeters is produced during use.

19. The device according to claim 1, wherein the orthotic (10) has at least one fenestration, selected from the group of fenestrations consisting of a maxillary fenestration (600) and a mandibular fenestration (700), allowing the exposure of at least one incisal surface of at least one tooth through the orthotic (10).

20. The device according to claim 1, wherein a portion of the lingual and occlusal surface of at least one molar, selected from the group of molars consisting of the left first molar, the left second molar, the right first molar and the right second molar is not covered by the orthotic (10).

21. An anti-obstructive airway dental orthotic (10), comprising:
a rigid strut (300) having a strut body (370) of variable length extendable between a predetermined minimum length and a predetermined maximum length, an anterior end (301) having a rotable strut to mandible pivot (360), and a posterior end (302) having a rotable strut to maxilla pivot (350),
a maxillary retainer (100) for cooperating with and reversibly attaching to a plurality of maxillary teeth of a user, having a spacing section (140) that has an area of increased thickness such that in use, a minimum vertical separation of approximately between 2 and 4 millimeters is produced between a cusp of the user's maxillary premolar and a cusp of the user's opposing mandibular premolar, wherein the maxillary retainer (100) includes a first maxilla to strut pivot point (120) and a second maxilla to strut pivot point (125), each having a central axis of rotation about the respective pivot point (120, 125), having an anterior-posterior separation of about 4 millimeters measured center-to-center along an imaginary line drawn parallel to an occlusal surface of the maxillary retainer (100), lying in a plane such that the central axes of both pivot points (120, 125) are each equidistant from the plane of the occlusal surface of the maxillary retainer (100),
a mandibular retainer (200) for cooperating with and reversibly attaching to a plurality of mandibular teeth of the user, wherein the mandibular retainer (200) includes a mandible to strut pivot point (220) having a central axis of rotation about the respective pivot point (220),
wherein the central axis of rotation of the first maxilla to strut pivot point (120), the second maxilla to strut pivot point (125), and the mandible to strut pivot point (220) all lie in parallel planes,
the strut to maxilla pivot (350) rotably cooperates with at least one of the first maxilla to strut pivot point (120) and the second maxilla to strut pivot point (125) to rotably and reversibly connect the maxillary retainer (100) and the strut (300), and
the strut to mandible pivot (360) rotably cooperates with the mandible to strut pivot point (220) to rotably and reversibly connect the mandibular retainer (200) and the strut (300), such that in use, an anterior mandible advancement beyond a point of habitual occlusion of approximately between 3 and 8 millimeters is produced.

22. The device according to claim 21, wherein the maxillary retainer (100) spacing section (140) comprises an area of increased thickness such that in use, a minimum vertical separation is produced between a cusp of the user's maxillary premolar and a cusp of the user's opposing mandibular premolar of approximately 4 millimeters.

23. The device according to claim 21, wherein in use, an anterior mandible advancement beyond the point of habitual occlusion of approximately 7.5 millimeters is produced.

24. A method for the reduction of Obstructive Sleep Apnea (OSA), comprising the steps of:
a) performing a baseline evaluation of a patient's medical, dental and sleep history;
b) forming an anti-obstructive airway dental orthotic (10), comprising:
1) a rigid strut (300) having a strut body (370) of variable length extendable between a predetermined minimum length and a predetermined maximum length, an anterior end (301) having a rotable strut to mandible pivot (360), and a posterior end (302) having a rotable strut to maxilla pivot (350),
2) a maxillary retainer (100) for cooperating with and reversibly attaching to a plurality of maxillary teeth of a user, comprising an area of increased thickness such that in use, a minimum vertical separation of approximately 2-4 millimeters is produced between a cusp of the user's maxillary premolar and a cusp of the user's opposing mandibular premolar, wherein the maxillary retainer (100) includes a first maxilla to strut pivot point (120) and a second maxilla to strut pivot point (125), each having a central axis of rotation about the respective pivot point (120, 125), having an anterior-posterior separation of about 4 millimeters measured center-to-center along an imaginary line drawn parallel to an occlusal surface of the maxillary retainer (100), lying in a plane such that the central axes of both pivot points (120, 125) are each equidistant from the plane of the occlusal surface of the maxillary retainer (100),
3) a mandibular retainer (200) for cooperating with and reversibly attaching to a plurality of mandibular teeth of the user, wherein the mandibular retainer (200) includes a mandible to strut pivot point (220) having a central axis of rotation about the respective pivot point (220),
4) wherein the central axis of rotation of the at least a first maxilla to strut pivot point (120), the at least a second maxilla to strut pivot point (125), and the mandible to strut pivot point (220) all lie in parallel planes,
5) the strut to maxilla pivot (350) rotably cooperates with at the second maxilla to strut pivot point (125) to rotably and reversibly connect the maxillary retainer (100) and the strut (300), and
6) the strut to mandible pivot (360) rotably cooperates with the mandible to strut pivot point (220) to rotably and reversibly connect the mandibular retainer (200) and the strut (300), such that in use, with the maxillary retainer (100) cooperating with and reversibly attached to the plurality of maxillary teeth of the user and the mandibular retainer (200) cooperating with and reversibly attached to the plurality of mandibular teeth of the user, an anterior mandible advancement of approximately 4 millimeters from a point of habitual occlusion is produced;
c) reversibly applying the orthotic (10) to the maxillary and mandibular teeth of the patient;
d) verifying the fit and comfort of the orthotic (10) to the patient;
e) waiting a predetermined period of time and making periodic assessments of a constellation of the patient's subjective symptomology;
f) waiting a predetermined period of time, and making an assessment of the constellation of the patient's objective symptomology;
g) evaluating and adjusting the thickness of the spacing area (140);
g) moving the strut (300) from the second maxilla to strut pivot point (125) to the first maxilla to strut pivot point (120), such that in use, with the maxillary retainer (100) cooperating with and reversibly attached to the plurality of maxillary teeth of the user and the mandibular retainer (200) cooperating with and reversibly attached to the plurality of mandibular teeth of the user, an anterior mandible advancement of approximately 7.5 millimeters from the point of habitual occlusion is produced;
i) waiting a predetermined period of time and making periodic assessments of a constellation of the patient's subjective symptomology;
j) waiting a predetermined period of time, and making an assessment of the constellation of the patient's objective symptomology;
k) repeating steps c-j as required for satisfactory relief of symptomology.
